(19) United States
(12) Patent Application Publication
(10) Pub. No.: US 2021/0278390 A1
(43) Pub. Date: Sep. 9, 2021

(54) ONLINE ANALYSIS OF OIL SANDS TAILINGS CONTENT

(71) Applicant: Dow Global Technologies LLC, Midland, MI (US)

(72) Inventors: Paul A. Gillis, Lake Jackson, TX (US); Jason S. Moore, Walnut Creek, CA (US); Michael K. Poindexter, Sugar Land, TX (US)

(21) Appl. No.: 16/333,857

(22) PCT Filed: Oct. 4, 2017
(86) PCT No.: PCT/US2017/055019

§ 371 Date: Mar. 15, 2019

Related U.S. Application Data
(60) Provisional application No. 62/415,192, filed on Oct. 31, 2016.

(51) Int. Cl. G01N 33/28; G01N 33/24; G01N 11/14; C10G 1/04

(52) U.S. Cl. CPC G01N 33/2823; G01N 33/24; G01N 2011/0053; C10G 1/045; G01N 11/14

(57) ABSTRACT

The present invention relates to a method for determining the clay content of an oil sands tailings stream. The method comprises treating an oil sands tailings stream with a PEO flocculant, passing the mixture through a dynamic mixer comprising a mixer shaft and a power drive for rotating said shaft, wherein the power input needed to maintain a mixer rotational speed (RPM) is measured. For an oil sands tailings stream having a specified solids content, the power measurement and PEO dosage level is correlated to a clay content characterization index, for example methylene blue index (MBI), to determine in real-time the clay content during the processing of an oil sands tailings stream.

### FIELD OF THE INVENTION

The disclosure relates generally to the field of oil sands processing. More particularly, the disclosure relates to a method for determining the clay content of an oil sands tailings stream.

### BACKGROUND OF THE INVENTION

Modern society is greatly dependent on the use of hydrocarbon resources for fuels and chemical feedstocks. Hydrocarbons are generally found in subsurface formations that can be termed “reservoirs”. Removing hydrocarbons from the reservoirs depends on numerous physical properties of the subsurface formations, such as the permeability of the rock containing the hydrocarbons, the ability of the hydrocarbons to flow through the subsurface formations, and the proportion of hydrocarbons present, among other things. Easily harvested sources of hydrocarbons are dwindling, leaving less accessible sources to satisfy future energy needs. As the costs of hydrocarbons increase, the less accessible sources become more economically attractive.

Recently, the harvesting of oil sands to remove heavy oil has become more economical. Hydrocarbon removal from oil sands may be performed by several techniques. For example, a well can be drilled to an oil sands reservoir and steam, hot air, solvents, or a combination thereof, can be injected to release the hydrocarbons. The released hydrocarbons may be collected by wells and brought to the surface. In another technique, strip or surface mining may be performed to access the oil sand, which can be treated with water, steam or solvents to extract the heavy oil.

Oil sands extraction processes are used to liberate and separate bitumen from oil sands so that the bitumen can be further processed to produce synthetic crude oil or mixed with diluent to form “dilbit” and be transported to a refinery plant. Numerous oil sands extraction processes have been developed and commercialized, many of which involve the use of water as a processing medium. Where the oil sands is treated with water, the technique may be referred to as water-based extraction (WBE). WBE is a commonly used process to extract bitumen from mined oil sand. Other processes are non-aqueous solvent-based processes. An example of a solvent-based process is described in Canadian Patent Application No. 2,724,806 (Adeyinka et al, published Jun. 30, 2011 and entitled “Process and Systems for Solvent Extraction of Bitumen from Oil Sands”), which is incorporated herein by reference. Solvent may be used in both aqueous and non-aqueous processes.

One WBE process is the Clark hot water extraction process (the “Clark Process”). This process typically requires that mined oil sands be conditioned for extraction by being crushed to a desired lump size and then combined with hot water and perhaps other agents to form a conditioned slurry of water and crushed oil sand. In the Clark Process, an amount of sodium hydroxide (caustic) may be added to the slurry to increase the slurry pH, which enhances the liberation and separation of bitumen from the oil sand. Other WBE processes may use other temperatures and may include other conditioning agents, which are added to the oil sands slurry, or may operate without conditioning agents. This slurry is first processed in a Primary Separation Cell (PSC), also known as a Primary Separation Vessel (PSV), to extract the bitumen from the slurry.

In one bitumen extraction process, a water and oil sands slurry is separated into three major streams in the PSC: bitumen froth, middlings, and a PSC underflow (also known as coarse tailings or primary separation tailings).

Regardless of the type of WBE process employed, the process will typically result in the production of a bitumen froth that requires treatment with a solvent. For example, in the Clark Process, a bitumen froth stream comprises bitumen, solids, and water. Certain processes use naphtha to dilute bitumen froth before separating the product bitumen by centrifugation. These processes are called naphthenic froth treatment (NFT) processes. Other processes use paraffinic solvent, and are called paraffinic froth treatment (PFT) processes, to produce pipelineable bitumen with low levels of solids and water. In the PFT process, a paraffinic solvent (for example, a mixture of iso-pentane and n-pentane) is used to dilute the froth before separating the product, diluted bitumen, by gravity. A portion of the asphaltenes in the bitumen is also rejected by design in the PFT process, and this rejection is used to achieve reduced solids and water levels. In both the NFT and the PFT processes, the diluted tailings (comprising water, solids and some hydrocarbon) are separated from the diluted product bitumen.

Solvent is typically recovered from the diluted product bitumen component before the bitumen is delivered to a refining facility for further processing.

The PFT process may comprise at least three units: Froth Separation Unit (FSU), Solvent Recovery Unit (SRU) and Tailings Solvent Recovery Unit (TSRU). Mixing of the solvent with the feed bitumen froth may be carried out counter-currently in two stages in separate froth separation units. The bitumen froth comprises bitumen, water, and solids. A typical composition of bitumen froth is about 60 wt % bitumen, 30 wt % water, and 10 wt % solids. The paraffinic solvent is used to dilute the froth before separating the product bitumen by gravity. The foregoing is only an example of a PFT process, and the values are provided by way of example only. An example of a PFT process is described in Canadian Patent No. 2,587,166 to Sury, which is incorporated herein by reference.

From the PSC, the middlings, comprising bitumen and about 10 to 30 wt % solids, or about 20 to 25 wt % solids, based on the total wt % of the middlings, is withdrawn and sent to the flotation cells to further recover bitumen. The middlings are processed by bubbling air through the slurry and creating a bitumen froth, which is recycled back to the PSC. Flotation tailings (FT) from the flotation cells, comprising mostly solids and water, are sent for further treatment or disposed in an external tailings area (ETA).

In ETA tailings ponds, a liquid suspension of oil sands fines in water with a solids content greater than 2 wt %, but less than the solids content corresponding to the Liquid Limit are called Fluid Fine Tailings (FFT). FFT settle over time to produce Mature Fine Tailings (MFT), having above about 30 weight percent solids.

It would be desirable to have an alternative or improved method of processing oil sands tailings.

### BRIEF SUMMARY OF THE INVENTION

The present invention is a method of determining the clay content of an aqueous mineral stream having a specified solids content, the method comprising the steps of: (i) contacting the aqueous mineral stream with an amount (in mg/L) of flocculating composition comprising a PEO to form a mixture, (ii) passing said mixture through a dynamic mixer having a mixing shaft with one or more impeller and a power drive having a power source wherein said drive rotates said shaft at a fixed or variable speed quantified by rotations per minute (RPMs), (iii) measuring a power input (P, in kW) to the power source required to rotate said mixer shaft at said RPMs, and (iv) correlating the power input to a clay content characterization index, preferably methylene blue index (MBI) value, using a statistical model to provide the clay content.

In one embodiment of the method described herein above, the determination is effected online, inline, or at line.

In one embodiment of the method described herein above, the determination is effected on a slipstream of the aqueous mineral stream.

In one embodiment of the method described herein above, the aqueous mineral stream is an aqueous oil sands tailings stream, preferably the oil sands tailings stream comprises coarse tailings stream, middlings, flotation tailings, froth separation tailings, tailings solvent recovery unit (TSRU) tailings, fluid fine tailings (FFT), mature fine tailings (MFT), thickened tailings, thickener overflow, centrifuged tailings, hydrocycloned tailings, or a combination thereof.

One embodiment of the method described herein above further comprises the step of (v) applying the determined clay content to adjust the flocculent composition dosage, preferably this step is effected automatically or manually.

Another embodiment of the method described herein above further comprises the step of: (vi) applying the determined clay content to adjust the aqueous mineral stream flow rate, preferably this step is effected automatically or manually.

### BRIEF DESCRIPTION OF THE DRAWINGS

FIG. 1 is a schematic of an embodiment of the method of the present invention.

FIG. 2 is a schematic plain view of a dynamic mixer apparatus of one embodiment of the method of the present invention.

### DETAILED DESCRIPTION OF THE INVENTION

At the outset, for ease of reference, certain terms used in this application and their meaning as used in this context are set forth below. To the extent a term used herein is not defined below, it should be given the broadest definition persons in the pertinent art have given that term as reflected in at least one printed publication or issued patent. Further, the present processes are not limited by the usage of the terms shown below, as all equivalents, synonyms, new developments and terms or processes that serve the same or a similar purpose are considered to be within the scope of the present disclosure.

Throughout this disclosure, where a range is used, any number between or inclusive of the range is implied.

A “hydrocarbon” is an organic compound that primarily includes the elements of hydrogen and carbon, although nitrogen, sulfur, oxygen, metals, or any number of other elements may be present in small amounts. Hydrocarbons generally refer to components found in heavy oil or in oil sand. However, the techniques described are not limited to heavy oils but may also be used with any number of other reservoirs to improve gravity drainage of liquids. Hydrocarbon compounds may be aliphatic or aromatic, and may be straight chained, branched, or partially or fully cyclic.

“Bitumen” is a naturally occurring heavy oil material. Generally, it is the hydrocarbon component found in oil sand. Bitumen can vary in composition depending upon the degree of loss of more volatile components. It can vary from a very viscous, tar-like, semi-solid material to solid forms. The hydrocarbon types found in bitumen can include aliphatics, aromatics, resins, and asphaltenes. A typical bitumen might be composed of:
19 weight (wt) % aliphatics (which can range from 5 wt % to 30 wt %, or higher);
19 wt % asphaltenes (which can range from 5 wt % to 30 wt %, or higher);
30 wt % aromatics (which can range from 15 wt % to 50 wt %, or higher);
32 wt % resins (which can range from 15 wt % to 50 wt %, or higher); and some amount of sulfur (which can range in excess of 7 wt %), the wt % based upon total weight of the bitumen.
   In addition, bitumen can contain some water and nitrogen compounds ranging from less than 0.4 wt % to in excess of 0.7 wt %. The percentage of the hydrocarbon found in bitumen can vary. The term “heavy oil” includes bitumen as well as lighter materials that may be found in a sand or carbonate reservoir.

“Heavy oil” includes oils which are classified by the American Petroleum 10 Institute (“API”), as heavy oils, extra heavy oils, or bitumens. The term “heavy oil” includes bitumen. Heavy oil may have a viscosity of about 1,000 centipoise (cP) or more, 10,000 cP or more, 100,000 cP or more, or 1,000,000 cP or more at ambient temperature. In general, a heavy oil has an API gravity between 22.3° API (density of 920 kilograms per meter cubed (kg/m³) or 0.920 grams per centimeter cubed (g/cm³)) and 10.0° API (density of 1,000 kg/m³ or 1 g/cm³). An extra heavy oil, in general, has an API gravity of less than 10.0° API (density greater than 1,000 kg/m³ or 1 g/cm³). For example, a source of heavy oil includes oil sands or bituminous sand, which is a combination of clay, sand, water and bitumen.

The term “bituminous feed” refers to a stream derived from oil sands that requires downstream processing in order to realize valuable bitumen products or fractions. The bituminous feed is one that comprises bitumen along with undesirable components. Undesirable components may include but are not limited to clay, minerals, coal, debris and water. The bituminous feed may be derived directly from oil sand, and may be, for example, raw oil sands ore. Further, the bituminous feed may be a feed that has already realized some initial processing but nevertheless requires further processing. Also, recycled streams that comprise bitumen in combination with other components for removal as described herein can be included in the bituminous feed. A bituminous feed need not be derived directly from oil sand, but may arise from other processes. For example, a waste product from other extraction processes which comprises bitumen that would otherwise not have been recovered may be used as a bituminous feed.

“Fine particles” or “fines” are generally defined as those solids having a size (i.e., diameter) of less than 44 microns (μm), as determined by laser diffraction particle size measurement.

“Coarse particles” are generally defined as those solids having a size (i.e., diameter) of greater than 44 microns (μm).

The term “solvent” as used in the present disclosure should be understood to mean either a single solvent, or a combination of solvents.

The terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numeral ranges provided. Accordingly, these terms should be interpreted as indicating that insubstantial or inconsequential modifications or alterations of the subject matter described and are considered to be within the scope of the disclosure.

The articles “the”, “a” and “an” are not necessarily limited to mean only one, but rather are inclusive and open ended so as to include, optionally, multiple such elements.

The term “paraffinic solvent” (also known as aliphatic) as used herein means solvents comprising normal paraffins, isoparaffins or blends thereof in amounts greater than 50 wt %. The presence of other components such as olefins, aromatics or naphthenes may counteract the function of the paraffinic solvent and hence may be present in an amount of only 1 to 20 wt % combined, for instance no more than 3 wt %. The paraffinic solvent may be a C₄ to C₂₀ or C₄ to C₆ paraffinic hydrocarbon solvent or a combination of iso and normal components thereof. The paraffinic solvent may comprise pentane, iso-pentane, or a combination thereof. The paraffinic solvent may comprise about 60 wt % pentane and about 40 wt % iso-pentane, with none or less than 20 wt % of the counteracting components referred above.

The present disclosure provides a method of analyzing a mineral tailings stream, preferably an oil sands tailings stream of the oil sands tailings treatment process to obtain a stream parameter.

The “oil sands tailings stream” may be any suitable stream stemming from oil sand. Examples include, but are not limited to, coarse tailings (also known as primary separation tailings), middlings, flotation tailings, froth separation tailings, tailings solvent recovery unit (TSRU) tailings, fluid fine tailings (FFT), mature fine tailings (MFT), thickened tailings, thickener overflow, centrifuged tailings, hydrocycloned tailings, or a combination thereof. The oil sands tailings stream may stem from aqueous based extraction. The oil sands tailings stream may stem from solvent based extraction.

The “oil sands tailings treatment process” means a process used to treat an oil sands tailings stream. Examples of treatment are vast and may include removing bitumen, solvent, or water.

A stream parameter means a parameter of the oil sands stream that is obtained by analyzing the oil sands tailings stream. The stream parameter determined in the method of the present invention is a clay parameter which corresponds to the clay content in the oil sands tailings stream. The clay content may comprise a weight percentage of particles that are clay in the oil sands tailings stream.

In the method of the present invention, the clay content is determined using a polyethylene oxide (PEO) flocculant. A mineral stream, preferably an oil sands tailings stream is blended with a PEO flocculant, the mixture enters a dynamic mixer comprising a mixer shaft having one or more impeller and a power drive for rotating said shaft, wherein the power input needed to maintain a prescribed mixer rotational speed (RPM) can be measured. For an oil sands tailings stream having a specified solids content, the power measurement (P), in kW along with the aqueous PEO flocculant dosage level [PEO], in mg/L may be correlated to a predetermined clay content characterization index, preferably methylene blue index (MBI), which characterizes the clay content in the oil sands tailings stream.

One embodiment of the present invention is a method for determining the clay content of an aqueous mineral stream having a specified solids content, preferably an aqueous oil sands tailings stream, comprising the steps of (i) contacting the aqueous mineral stream having a specified solids content with an amount of flocculating composition comprising a PEO (in mg/L) to form a mixture, (ii) passing said mixture through a dynamic mixer having a mixing shaft with one or more impeller and a power drive having a power source wherein said drive rotates said shaft at a fixed or variable speed quantified by rotations per minute (RPMs), (iii) measuring a power input (P in kW) to the power source required to rotate said mixer shaft at said RPMs, and (iv) correlating the power input to a clay content characterization index, preferably methylene blue index (MBI) value, wherein the statistical model to provide the clay content is dependent on the specific geometry of the mixer, i.e., mixer diameter, number of stages (impellers), size and/or shape of the impellers, etc.

One reason the ability to determine real-time the clay content is useful is that the clay content may vary considerably depending on the geological properties of the location that is being mined. The MBI can swing significantly on an hourly basis as MFT is dredged up from a pond. The effective amount of flocculant used to treat the tailings depends on the clay content and overtreatment is both costly and often gives poor performance. Hence, the operator needs to know the clay instantaneously so the flocculant additive level can be changed corresponding to the MBI level.

The flocculant composition of the method of the present invention comprises a polymeric flocculant, preferably a poly(ethylene oxide) homopolymer, a poly(ethylene oxide) copolymer, or mixtures thereof, herein after collectively referred to as “poly(ethylene oxide) (co)polymer”. Poly(ethyleneoxide) (co)polymers and methods to make said polymers are known, for example see WO 2013116027, WO2016/019213, and WO2016/019214, all of which are incorporated herein by reference in their entirety. In one embodiment of the present invention, a zinc catalyst, such as disclosed in U.S. Pat. No. 4,667,013, can be employed to make the poly(ethylene oxide) (co)polymers of the present invention. In a preferred embodiment the catalyst used to make the poly(ethylene oxide) (co)polymers of the present invention is a calcium catalyst such as those disclosed in U.S. Pat. Nos. 2,969,402; 3,037,943; 3,627,702; 4,193,892; and 4,267,309, all of which are incorporated by reference herein in their entirety.

Suitable poly(ethylene oxide) homopolymers and/or poly(ethylene oxide) copolymers useful in the method of the present invention have a weight average molecular weight equal to or greater than 100,000 daltons (Da) and equal to or less than 15,000,000 Da, preferably equal to or greater than 1,000,000 Da and equal to or less than 8,000,000 Da.

Suitable amounts of the flocculant composition comprising the poly(ethylene oxide) (co)polymer to be added to the mineral suspensions range from 10 grams to 10,000 grams per ton of mineral solids. Generally the appropriate dose can vary according to the particular material composition (i.e., clay content) and material solids content. Preferred doses are in the range 30 to 7,500 grams per ton, more preferably 100 to 3,000 grams per ton, while even more preferred doses are in the range from 500 to 3,000 grams per ton. The flocculant composition comprising a poly(ethylene oxide) (co)polymer may be added to the suspension of particulate mineral material, e.g., the tailings slurry, in solid particulate form, an aqueous solution that has been prepared by dissolving the poly(ethylene oxide) (co)polymer into water, or an aqueous-based medium, or a suspended slurry in a solvent.

In the method of the present invention, the flocculant composition comprising a poly(ethylene oxide) (co)polymer may further comprise one or more other types of flocculant (e.g., polyacrylates, polymethacrylates, polyacrylamides, partially-hydrolyzed polyacrylamides, cationic derivatives of polyacrylamides, polydiallyldimethylammonium chloride (pDADMAC), copolymers of DADMAC, cellulosic materials, chitosan, sulfonated polystyrene, linear and branched polyethyleneimines, polyvinylamines, etc.) or other type of additive typical for flocculant compositions.

Coagulants, such as salts of calcium (e.g., gypsum, calcium oxide, and calcium hydroxide), aluminum (e.g., aluminum chloride, sodium aluminate, and aluminum sulfate), iron (e.g., ferric sulfate, ferrous sulfate, ferric chloride, and ferric chloride sulfate), magnesium carbonate, other multi-valent cations and pre-hydrolyzed inorganic coagulants, may also be used in conjunction with the poly(ethylene oxide) (co)polymer.

The clay content determination may be combined with a slurry flow rate and on-line slurry density, or a solids content of the oil sands stream, to obtain a mass flow rate parameter comprising a clay mass flow rate parameter.

The clay content determination may be used in a second step to adjust flocculant composition dosage, flocculant dosage, coagulant dosage, flocculent mixing equipment operation, downstream thickener operation, or blending ratio with another oil sands tailings stream or a dilution water stream. Adjustment of flocculent dosage may be particularly useful or convenient.

In one embodiment, the second step may comprise adjustment of flocculent dosage to a thickened tailings stream during a re-flocculation step.

In one embodiment, the second step may comprise adjustment of a flow rate of the oil sands tailings stream.

The analyzed oil sands tailings stream may be at least one of three streams, a flotation or TSRU (tailings solvent recovery unit) tailings stream, a coarse tailings stream, and a fluid fine tailings stream.

The analyzed oil sands tailings stream may be at least one of a feed stream to a thickener, a thickener overflow, and a thickener underflow.

The analysis is effected online, inline, or at line. Operation in real-time may be advantageous.

The clay content determination analysis may be effected on a slipstream of the oil sands tailings stream. The slipstream may be a representative sample of the stream and may be on a vertical section of a pipe or after a pump.

While the stream parameter may be used to adjust the process, the parameter may also be converted to another measurement, which can provide useful information and which can in turn be used to adjust a process parameter.

The process adjustment may be any suitable process adjustment. The process adjustment may be adjustment of polymer dosage, caustic dosage, or blending ratio with another oil sands stream. The process adjustment may be adjustment to achieve a sands to fines ratio (SFR) of a resultant tailings stream within a predetermined range. The process adjustment may be adjustment of a flocculant addition rate. The process adjustment may be adjustment to achieve 0 to 44 μm particle content of a hydrotransport slurry within a predetermined range. The process adjustment may be adjustment of a caustic dosage to a hydrotransport slurry based on reference data. The reference data may be experimental data or otherwise.

Typically, the material to be flocculated may be derived from or contain filter cake, tailings, thickener underflows, or unthickened plant waste streams, for instance other mineral tailings, slurries, or slimes, including phosphate, diamond, gold slimes, mineral sands, tails from zinc, lead, copper, silver, uranium, nickel, iron ore processing, coal, oil sands or red mud. The material may be solids settled from the final thickener or wash stage of a mineral processing operation. Thus the material desirably results from a mineral processing operation. Preferably the material comprises tailings. Preferably the mineral material would be selected from red mud and tailings containing clay, such as oil sands tailings, etc.

The oil sands tailings or other mineral suspensions may have a solids content in the range 5 percent to 80 percent by weight. The slurries or suspensions often have a solids content in the range of 10 percent to 70 percent by weight, for instance 25 percent to 40 percent by weight. The sizes of particles in a typical sample of the fine tailings are substantially all less than 45 microns, for instance about 95 percent by weight of material is particles less than 20 microns and about 75 percent is less than 10 microns. The coarse tailings are substantially greater than 45 microns, for instance about 85 percent is greater than 100 microns but generally less than 10,000 microns. The fine tailings and coarse tailings may be present or combined together in any convenient ratio provided that the material remains pumpable.

The dispersed particulate solids may have a unimodal, bimodal, or multimodal distribution of particle sizes. The distribution will generally have a fine fraction and a coarse fraction, in which the fine fraction peak is substantially less than 44 microns and the coarse (or non-fine) fraction peak is substantially greater than 44 microns.

In one embodiment, the present invention relates to a method for determining the clay content of an aqueous solution of sands tailings. As used herein, the term “tailings” means tailings derived from oil sands extraction operations and containing a fines fraction. The term is meant to include fluid fine tailings (FFT) and/or mature fine tailings (MFT) tailings from ongoing extraction operations (for example, thickener underflow or froth treatment tailings) which may bypass a tailings pond and from tailings ponds. The oil sands tailings will generally have a solids content of 10 to 70 weight percent, or more generally from 25 to 40 weight percent, and may be diluted to 20 to 25 weight percent with water for use in the present process.

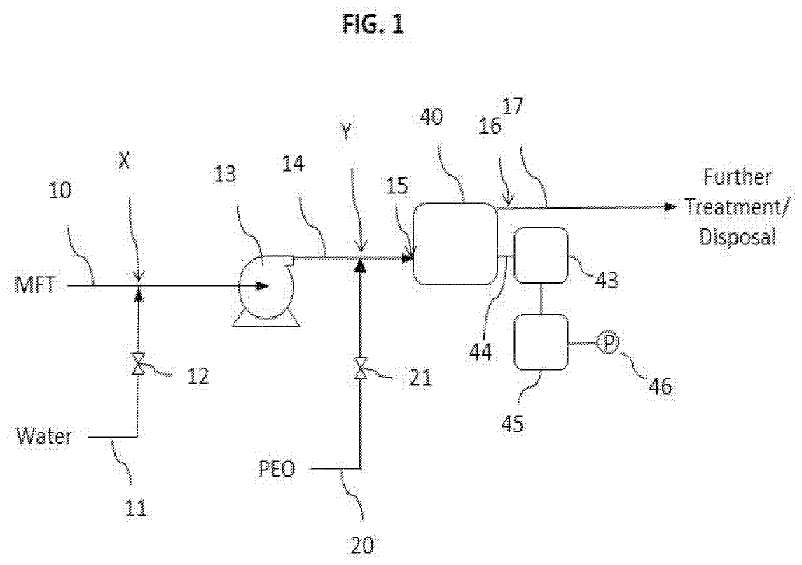

A schematic of an embodiment of the present invention is shown in FIG. 1. The aqueous suspension containing solids such as oil sands mature fine tailings (MFT) in line 10 is pumped via pump 13 through a transportation conduit, preferably a first pipeline, line 14. If desired, additional water can be added to the MFT through line 11 at Point X. The flocculant composition comprising a poly(ethylene oxide) (co)polymer (referred herein after to as “PEO”) is added to the aqueous MFT suspension and the MFT and PEO are mixed in-line to form a dough-like mixture, for example through line 20 at Point Y. In other embodiments of the method of the present invention, the flocculant composition may be added to the MFT suspension at any time prior to entering the in-line pipeline reactor 40. To facilitate blending and interactions between the MFT and the PEO the combined stream can flow through a pipeline optionally containing a static mixing device, such as an in-line static mixer, or the like (not shown in the drawings) may be located in the first pipeline 14 after the addition point of the PEO Y and before the in-line pipeline reactor 40.

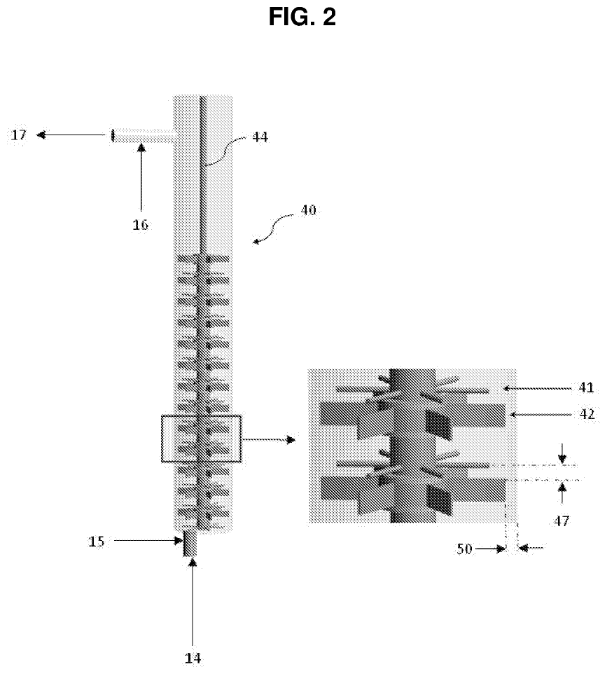

The dough-like mixture enters an in-line pipeline reactor 40. The pipeline reactor 40 comprises one or more rotor 41, preferably in combination with one or more stator 42, FIG. 2. Preferably, one or more rotor 41 and one or more stator 42 are arranged in an alternating fashion, i.e., rotor, stator, rotor, stator, etc. It is understood that the size, location and number of rotors and/or stators used in the in-line dynamic mixer 40 is dependent upon the overall dimensions (volume) of the dynamic mixer necessary for a particular operation.

The special orientation, with regard to the ground, of the pipeline reactor 40 in the method of the present invention is not limited, it may be horizontal, vertical, or at any angle in between. Preferably the pipeline reactor 40 is in a vertical orientation wherein the dough-like mixture of MFT and PEO enters directly through line 14 at the bottom of the pipeline reactor 40 or optionally through the reactor inlet pipe 15 and then flows out the top of the pipeline reactor 40 directly into line 17 or optionally through the reactor outlet pipe 16 into line 17 for further treatment and/or transferred to a settling area for disposal.

In one embodiment of the present invention, the pipeline reactor 40 of the present invention may be a separate tank, a stirred tank reactor, a separation vessel, a batch vessel, a semi-batch vessel, or the like.

In one embodiment of the present invention, the pipeline reactor 40 of the present invention is not a separate tank, a stirred tank reactor, a separation vessel, a batch vessel, a semi-batch vessel, or the like.

In one embodiment of the present invention, the pipeline reactor 40 may have various components and configurations.

The addition stage for the introduction of the PEO into the aqueous solution of oil sands tailings comprises any suitable means for adding the PEO, for example an injector quill, a single or multi-tee injector, an impinging jet mixer, a sparger, a multi-port injector, and the like. The flocculant composition comprising a poly(ethylene oxide) (co)polymer is added as a solid, slurry, or dispersion, preferably an aqueous solution. The addition stage is herein after referred to as in-line addition. The in-line addition of the PEO occurs through line 20 at point Y under conditions which exclude dynamic mixing, in other words, the addition occurs without mechanical energy input (i.e., moving parts) at the point of initial contacting of the two feeds. The PEO injection point can be before or within a static mixer or into the pipeline. In one embodiment, the mixing is facilitated by the presence of a secondary pump (e.g., a progressive cavity pump) or an in-line static mixer (neither shown in the figures) downstream from the injector in the direction of flow from where the PEO is added.

After the flocculant composition comprising a poly(ethylene oxide) (co)polymer is added and begins to mix with the oil sands tailing suspension a viscous, dough-like mixture is formed.

The rotors 41 are connected to a mixer shaft 44 which is rotated by a drive 43 to provide shear to the dough-like mixture of MFT and PEO. In one embodiment, said drive is provided at the opposite end from where the dough-like mixture enters the in-line reactor, may be, for example a variable speed motor or constant speed motor. The drive is powered by a power source 45 having a mechanism 46, such as a gauge, from which the amount of energy needed to rotate the shaft 44 can be determined.

It should be understood that numerous changes, modifications, and alternatives to the preceding disclosure can be made without departing from the scope of the disclosure. The preceding description, therefore, is not meant to limit the scope of the disclosure. Rather, the scope of the disclosure is to be determined only by the appended claims and their equivalents. It is also contemplated that structures and features in the present examples can be altered, rearranged, substituted, deleted, duplicated, combined, or added to each other.

### EXAMPLES

### Continuous Flow MFT Treatment.

MFT samples (with varied wt % solids and MBI) are treated in a continuous process. A 0.4 wt % aqueous solution of a poly(ethylene oxide) homopolymer having a weight average molecular weight of 8,000,000 Da available as POLYOX™ WSR 308 poly(ethylene oxide) polymer (WSR 308) from The Dow Chemical Company is pumped into an MFT flow to give a range of polymer dosing. The polymer solution is added upstream of a progressive cavity pump used to control the MFT flow rate. This mixed stream, flowing at 10 gpm, is then directed into a dynamic mixing apparatus operating at a range of rotational speeds. Table 1 lists several experimental conditions and the resulting power readings. A statistical analysis of the results shows that the dynamic mixer rotational speed (RPM), polymer concentration, and MBI have significant effects on the measured power. This statistical model produced the following relationship between these variables:

Power (in kW)=0.7472385 log(RPM)+0.0523667 MBI+0.1283805 log(polymer concentration in mg/L)−5.520894

The statistical model of the data when the mixer RPMs are fixed at 900 RPM is as follows:

Power (in kW)=0.051585 MBI+0.128087 log(polymer concentration in mg/L−0.434348

The majority of the data shown is for a small range of MFT solids wt %. In field application, a larger range of solids may be dredged from the existing tailings ponds. In one embodiment of the method of the present invention, to account for this effect, an online densitometer may be used to determine this solids content in real-time and accounted for in the power analysis with appropriate calibration.

In another embodiment of the method of the present invention, if no mixing is needed to produce the desired dewatering performance, a small slipstream from the process may be taken for this analysis and then used as feedback to control process variables as necessary to appropriately treat feeds of changing clay and solids content.

TABLE 1

| | MFT Solids, | | | Polymer, | Mixer Power, |
|---|---|---|---|---|---|
| Example | wt % | RPM | MBI | ppm | kW |
| 1 | 41.6 | 900 | 7.7 | 1150 | 0.740 |
| 2 | 41.6 | 500 | 7.7 | 1150 | 0.320 |
| 3 | 41.6 | 900 | 7.7 | 504 | 0.670 |
| 4 | 41.6 | 500 | 7.7 | 501 | 0.290 |
| 5 | 41.6 | 900 | 7.7 | 303 | 0.640 |
| 6 | 41.6 | 900 | 7.7 | 120 | 0.540 |
| 7 | 38.0 | 900 | 8.0 | 135 | 0.560 |
| 8 | 38.0 | 900 | 8.0 | 72 | 0.400 |
| 9 | 38.0 | 900 | 8.0 | 37 | 0.440 |
| 10 | 38.0 | 900 | 8.0 | 213 | 0.560 |
| 11 | 38.0 | 900 | 8.0 | 98 | 0.480 |
| 12 | 41.6 | 900 | 7.7 | 140 | 0.531 |
| 13 | 41.4 | 900 | 9.2 | 152 | 0.503 |
| 14 | 41.4 | 1500 | 9.2 | 153 | 1.037 |
| 15 | 41.4 | 900 | 9.2 | 153 | 0.557 |
| 16 | 41.4 | 500 | 9.2 | 154 | 0.199 |
| 17 | 41.4 | 900 | 9.2 | 321 | 0.684 |
| 18 | 41.4 | 500 | 9.2 | 313 | 0.223 |
| 19 | 39.8 | 900 | 7.6 | 173 | 0.482 |
| 20 | 38.7 | 900 | 10.0 | 137 | 0.561 |
| 21 | 38.7 | 900 | 10.0 | 339 | 0.804 |
| 22 | 38.7 | 900 | 10.0 | 2482 | 1.012 |
| 23 | 40.0 | 900 | 7.8 | 135 | 0.488 |
| 24 | 40.0 | 900 | 7.8 | 48 | 0.395 |
| 25 | 40.7 | 900 | 7.4 | 154 | 0.530 |
| 26 | 32.4 | 900 | 7.4 | 169 | 0.378 |

What is claimed is:

1. A method of determining the clay content of an aqueous mineral stream having a specified solids content, the method comprising the steps of:
(i) contacting the aqueous mineral stream with an amount (in mg/L) of flocculating composition comprising a PEO to form a mixture,
(ii) passing said mixture through a dynamic mixer having a mixing shaft with one or more impeller and a power drive having a power source wherein said drive rotates said shaft at a fixed or variable speed quantified by rotations per minute (RPMs),
(iii) measuring a power input (P, in kW) to the power source required to rotate said mixer shaft at said RPMs,
and
(iv) correlating the power input to a clay content characterization index using a statistical model to provide the clay content.

2. The method of claim 1 wherein the clay content characterization index is a methylene blue index (MBI) value.

3. The method of claim 1 is effected online, inline, or at line.

4. The method of claim 1 is effected on a slipstream of the aqueous mineral stream.

5. The method of claim 1, wherein the aqueous mineral stream is an aqueous oil sands tailings stream.

6. The method of claim 5, wherein the oil sands tailings stream comprises coarse tailings stream, middlings, flotation tailings, froth separation tailings, tailings solvent recovery unit (TSRU) tailings, fluid fine tailings (FFT), mature fine tailings (MFT), thickened tailings, thickener overflow, centrifuged tailings, hydrocycloned tailings, or a combination thereof.

7. The method of any one of claim 1 further comprising the step of
(v) applying the determined clay content to adjust the flocculent composition dosage.

8. The method of claim 1 further comprising the step of:
(vi) applying the determined clay content to adjust the aqueous mineral stream flow rate.

9. The method claim 7 step (v) is effected automatically or manually.

10. The method claim 8 step (vi) is effected automatically or manually.
